(19) United States
(12) Patent Application Publication
(10) Pub. No.: US 2021/0342900 A1
(43) Pub. Date: Nov. 4, 2021

(54) METHODS FOR CUSTOMIZED RULE ENGINES FOR AUTOMATED MEDICAL BILL REVIEW AND DEVICES THEREOF

(71) Applicant: Mitchell International, Inc., San Diego, CA (US)

(72) Inventors: Ashraf Memon, San Diego, CA (US); Suzi Strong, Pacifica, CA (US); Sean Boren, San Diego, CA (US); Mats Tigerstrom, San Diego, CA (US)

(21) Appl. No.: 16/866,375

(22) Filed: May 4, 2020

(51) Int. Cl. G06Q 30/04; G16H 15/00; G16H 40/20; G16H 80/00

(52) U.S. Cl. CPC G06Q 30/04; G16H 80/00; G16H 40/20; G16H 15/00

(57) ABSTRACT

Methods, non-transitory machine readable media, and bill review engine devices that provide customized rule engines for automated medical bill review are disclosed. With this technology, a selection of a reusable logical pattern from a pattern library is received. The reusable logical pattern comprises source code defining at least one action and one or more template fields. The template fields are populated based on received rule parameter data to generate and store a rule. The rule parameter data comprises one or more triggering codes. The rule is then applied to contents of a received electronic medical bill to determine when the rule is triggered based on the triggering codes. A pricing result for the electronic medical bill, determined based on the action, is subsequently returned to a source of the electronic medical bill, when the determination indicates that the rule is triggered.

### FIELD

This technology generally relates to customized rule engines for automated electronic medical bill review and repricing and, more particularly, to methods and devices for leveraging reusable logical patterns and improved graphical user interfaces to efficiently generate customized rules without requiring source code development.

### BACKGROUND

Insurance carriers or payers submit electronic medical bills, often in relatively large batches, for review to determine whether the bill should be disqualified or rejected or whether the associated fee is accurate or should be adjusted, for example according to factors such as jurisdictional regulations, proprietary edits, industry standard practices, correct coding, provider fraud, duplicate checks, billing errors, other payment calculations, and the like. The electronic medical bills can be workers compensation or auto casualty medical bills, for example, although other types of bills can be submitted to an automated review as well. The electronic medical bills are generally processed by a rule engine that applies rules that are highly dependent on the data extracted from the content of the electronic medical bills, for example according to factors such as pricing, pre-pricing, various data checks, scrubbing, editing, warning, pricing and other adjustment rules affecting the final outcome of the bill, and the like.

The government regulations that form the basis of the rules applied by the rule engine generally evolve and are released at a rapid pace, and rule engine users increasingly demand that changes in the industry and regulations are reflected in production rule engines promptly. However, current rule engines do not support efficient rule generation. In particular, business analysts often need to explain to software developers how the regulations should be reflected in the rule engine with respect to the associated applicable medical bill data and rule outcomes, and the software developers subsequently require a relatively significant amount of time to generate the source code for the new or modified rules that can be deployed.

Accordingly, current rule engines for automated electronic medical bill review are challenging to maintain, requiring software development expertise. Moreover, current rule engines have reduced effectiveness due to the slow responsiveness to evolving regulations.

### SUMMARY

A method for automated medical bill review is disclosed that includes receiving a selection of a reusable logical pattern from a pattern library. The reusable logical pattern comprises source code defining at least one action and one or more template fields. The template fields are populated based on received rule parameter data to generate and store a rule. The rule parameter data comprises one or more triggering codes. The rule is then applied to contents of a received electronic medical bill to determine when the rule is triggered based on the triggering codes. A pricing result for the electronic medical bill, determined based on the action, is subsequently returned to a source of the electronic medical bill, when the determination indicates that the rule is triggered.

A bill review engine device is disclosed that includes memory including programmed instructions stored thereon and one or more processors configured to execute the stored programmed instructions to receive a selection of a reusable logical pattern from a pattern library. The reusable logical pattern comprises source code defining at least one action and one or more template fields. The template fields are populated based on received rule parameter data to generate and store a rule. The rule parameter data comprises one or more triggering codes. The rule is then applied to contents of a received electronic medical bill to determine when the rule is triggered based on the triggering codes. A pricing result for the electronic medical bill, determined based on the action, is subsequently returned to a source of the electronic medical bill, when the determination indicates that the rule is triggered.

A non-transitory machine readable medium is disclosed that has stored thereon instructions for automated medical bill review including executable code that, when executed by one or more processors, causes the processors to receive a selection of a reusable logical pattern from a pattern library. The reusable logical pattern comprises source code defining at least one action and one or more template fields. The template fields are populated based on received rule parameter data to generate and store a rule. The rule parameter data comprises one or more triggering codes. The rule is then applied to contents of a received electronic medical bill to determine when the rule is triggered based on the triggering codes. A pricing result for the electronic medical bill, determined based on the action, is subsequently returned to a source of the electronic medical bill, when the determination indicates that the rule is triggered.

This technology has a number of associated advantages including providing methods, non-transitory computer readable media, and bill review engine devices that facilitate rapid generation of rules using reusable logical patterns and a customized graphical user interface for more effective, automated electronic medical bill review. This technology provides an improved rule engine that allows business analysts to generate rules, and other system output that communicates clear system explanations for each individual rule outcome provided by the rules, based on reusable logical patterns stored in a pattern library and without software developer interaction. The rule engine of this technology advantageously provides an audit trail and configurable outcomes with more informed reporting of disqualification and repricing decisions.

### BRIEF DESCRIPTION OF THE DRAWINGS

FIG. 1 a block diagram of an exemplary network environment with a bill review engine device;

FIG. 2 is a block diagram of an exemplary bill review engine device;

FIG. 3 is a flowchart of an exemplary method for generating reusable logical patterns and deploying rules generated from the reusable logical patterns;

FIG. 4 is a chart of exemplary rule contents;

FIG. 5 is a screenshot of an exemplary pattern search interface;

FIG. 6 is a screenshot of an exemplary rule header interface;

FIG. 7 is a screenshot of an exemplary rule trigger code interface;

FIG. 8 is a screenshot of an exemplary rule outcome interface;

FIG. 9 is a screenshot of an exemplary rule jurisdiction interface;

FIG. 10 is a screenshot of an exemplary rule order overlay; and

FIG. 11 is a flowchart of an exemplary method for executing an improved rule engine for automated electronic medical bill review.

### DETAILED DESCRIPTION

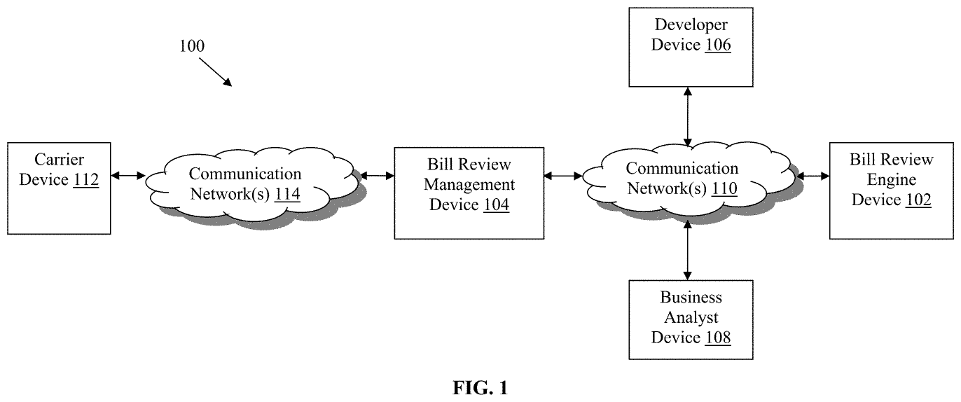

Referring to FIG. 1, an exemplary network environment 100 with an exemplary bill review engine device 102 is illustrated. The bill review engine device 102 in this example is coupled to a bill review management device 104, a developer device 106, and a business analyst device 108 via communication network(s) 110, and to a carrier device 112 via the communication network(s) 110, bill review management device 104, and communication network(s) 114. The bill review engine device 102, bill review management device 104, developer device 106, business analyst device 108, and carrier device 112 may be coupled together via other topologies in other examples. Additionally, the network environment 100 may include other network devices such as one or more routers and/or switches, for example, which are well known in the art and thus will not be described herein. This technology provides a number of advantages and technical improvements including methods, non-transitory computer readable media, and bill review engine devices that more efficiently facilitate rule generation using improved graphical user interfaces and stored reusable logical patterns to improve automated electronic medical bill analysis.

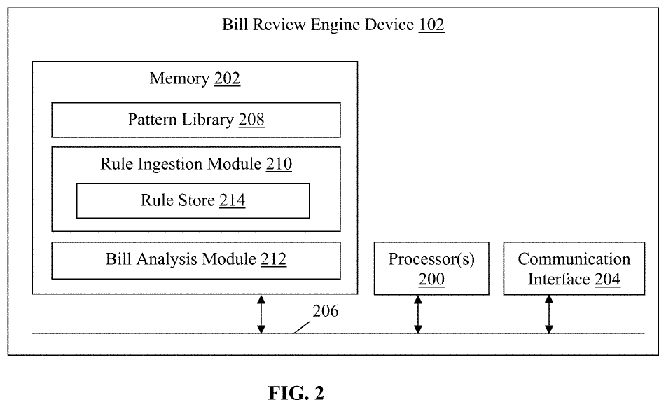

Referring to FIGS. 1-2, the bill review engine device 102 in this example includes one or more processors 200, a memory 202, and/or a communication interface 204, which are coupled together by a bus 206 or other communication link, although the bill review engine device 102 can include other types and/or numbers of elements in other configurations. The processor(s) 200 of the bill review engine device 102 may execute programmed instructions stored in the memory 202 for the any number of the functions described and illustrated herein. The processor(s) 200 may include one or more CPUs or general purpose processors with one or more processing cores, for example, although other types of processor(s) can also be used.

The memory 202 of the bill review engine device 102 stores these programmed instructions for one or more aspects of the present technology as described and illustrated herein, although some or all of the programmed instructions could also be stored elsewhere. A variety of different types of memory storage devices, such as random access memory (RAM), read only memory (ROM), hard disk, solid state drives (SSDs), flash memory, or other computer readable medium which is read from and written to by a magnetic, optical, or other reading and writing system that is coupled to the processor(s) 200, can be used for the memory 202.

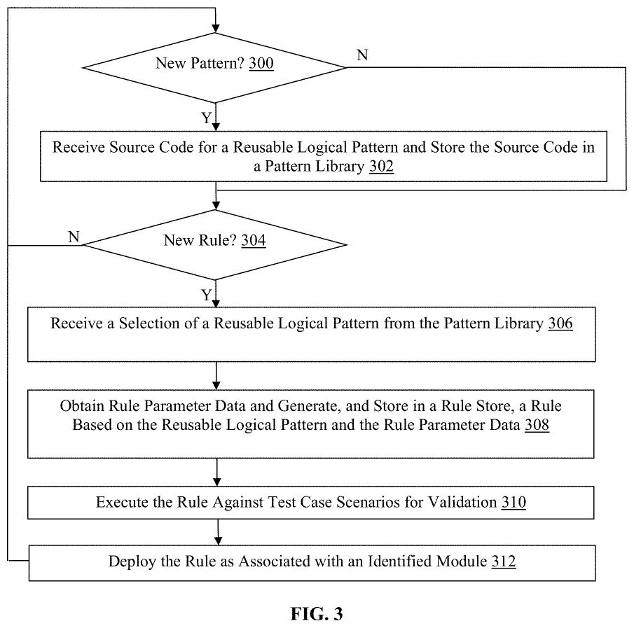

Accordingly, the memory 202 can store application(s) that can include executable instructions that, when executed by the bill review engine device 102, cause the bill review engine device 102 to perform actions, such as to transmit, receive, or otherwise process network messages, for example, and to perform other actions described and illustrated below with reference to FIGS. 3-11. The application(s) can be implemented as modules or components of other application(s). Further, the application(s) can be implemented as operating system extensions, module, plugins, or the like.

Even further, the application(s) may be operative in a cloud-based computing environment. The application(s) can be executed within or as virtual machine(s) or virtual server(s) that may be managed in a cloud-based computing environment. Also, the application(s), and even the bill review engine device 102 itself, may be located in virtual server(s) running in a cloud-based computing environment rather than being tied to one or more specific physical network computing devices. Also, the application(s) may be running in one or more virtual machines (VMs) executing on the bill review engine device 102. In one or more embodiments of this technology, virtual machine(s) running on the bill review engine device 102 may be managed or supervised by a hypervisor.

Some embodiments feature a sequential engine, especially when compared with hard-coded implementations. Such embodiments have numerous advantages. For example, and analyst may select the order of execution of the rules. A multi-threaded implementation allows multiple bills to be processed simultaneously, increasing throughput. Such embodiments are customer-independent, as opposed to previous implementations where a separate application was deployed for each customer. In these embodiments, each customer may provide a custom configuration to select only those rules that apply to the customer. Some implementations also feature interstitial logic to implement common-sense rules, for example to group three instances of a procedure together.

In this particular example, the memory 202 of the bill review engine device 102 includes a pattern library 208, a rule ingestion module 210, and a bill analysis module 212, although the memory 202 can include other policies, modules, databases, or applications, for example. The pattern library 208 in this example stores reusable logical patterns that are associated with unique identifiers and include source code generated by a user of the developer device 106. The reusable logical patterns include common logic that may be applicable to any number of rules that are associated with different jurisdictions, for example. In some examples, the reusable logical patterns include criteria including template fields and at least one action (e.g., to disqualify or reprice an electronic medical bill).

The rule ingestion module 210 in this example is configured to provide a graphical user interface (GUI) to the business analyst device and receive rule parameter data via the GUI for a particular rule that is built on top of one of the reusable logical patterns selected by a user of the business analyst device 108 from the pattern library 208. With the rule parameter data, the rule ingestion module 210 populates the template fields of selected reusable logical pattern to generate a rule having the associated action of the selected reusable logical pattern. The generates rules can be associated with particular jurisdictions (e.g., states) and/or modules (e.g., pre-pricing, pricing, and adjustment) and can be maintained in the rule store 214.

The bill analysis module 212 is configured to receive electronic medical bills from the bill review management device 104 and apply the appropriate rules retrieved from the rule store 214 to each of the electronic medical bills to generate a response. The bill review management device 104 can facilitate access to the response via a GUI provided to the carrier device 112, for example. The response can include a final pricing amount for one or more of the medical bills, an outcome description that explains how the repricing amount was generated, and/or an audit trail of the triggered rules, for example, among other types of information described and illustrated in more detail later.

The communication interface 204 of the bill review engine device 102 operatively couples and communicates between the bill review engine device 102, the bill review management device 104, developer device 106, business analyst device 108, and carrier device 112, which are all coupled together by the communication network(s) 110 and 114, although other types and/or numbers of communication networks or systems with other types and/or numbers of connections and/or configurations to other devices and/or elements can also be used.

By way of example only, the communication network(s) 110 and 114 can include local area network(s) (LAN(s)) or wide area network(s) (WAN(s)), and can use TCP/IP over Ethernet and industry-standard protocols, although other types and/or numbers of protocols and/or communication networks can be used. The communication network(s) 110 and 114 in this example can employ any suitable interface mechanisms and network communication technologies including, for example, Ethernet-based Packet Data Networks (PDNs) and the like.

The bill review engine device 102 can be a standalone device or integrated with one or more other devices or apparatuses, such as the bill review management device 104, for example. In one particular example, the bill review engine device 102 can include or be hosted by the bill review management device 104, and other arrangements can also be used in other examples.

The bill review management device in this example includes processor(s), a memory, and a communication interface, which are coupled together by a bus or other communication link, although other numbers and/or types of network devices could be used. The communication interface can be used to interface with the carrier device 112 via the communication network(s) 114, to obtain electronic medical bills and/or provide GUIs to facilitate analysis of the automated review of the electronic medical bills by the bill review engine device 102, for example, and with the bill review engine device 102 via the communication network(s) 110 to send electronic medical bills to the bill review engine device 102 and receive a response including a result of the electronic medical bill review.

The developer device 106, business analyst device 108, and carrier device 112 in this example include any type of computing device that can be used to interface with the bill review management device 104 or bill review engine device 102 to submit data and/or receive GUI(s), for example. Each of the developer device 106, business analyst device 108, and carrier device 112 in this example includes a processor, a memory, and a communication interface, which are coupled together by a bus or other communication link, although other numbers and/or types of network devices could be used. The developer device 106, business analyst device 108, and carrier device 112 may run interface application(s), such as standard web browsers or standalone client applications, which may provide an interface to communicate with the bill review management device 104 or bill review engine device 102 via the communication network(s) 110 and/or 114. The bill review management device 104 or bill review engine device 102 may further include a display device, such as a display screen or touchscreen, and/or an input device, such as a keyboard, for example.

The developer device 106 in this example is used by a software developer, for example, to generate the source code for the reusable logical patterns stored in the pattern library 208. The business analyst device 108 is used by a business analyst to generate rules built on top of the reusable logical patterns via GUIs provided by the bill review engine device 102. By leveraging the reusable logical patterns, the rules can advantageously be generated without any particular knowledge of, or expertise regarding, the underlying source code for the reusable logical patterns. Accordingly, the business analyst device 108 is used to rapidly respond to evolving government regulations that impact electronic medical bill review by efficiently creating or modifying rules applied by the bill analysis module and maintained in the rule store 214.

The carrier device 112 can be used by an insurance carrier or payer that requires an automated review of electronic medical bills that may be associated with insured customers, for example. The carrier device 112 can be used to facilitate communication of the electronic medical bills to the bill review management device 104, and ultimately the bill review engine device 102, although other methods for obtaining the electronic medical bills by the bill review management device 104 can also be used. The carrier device 112 also receives GUIs from the bill review management device 104 that allow a user to analyze the result of the automated medical bill review by the bill review engine device 102, including electronic medical bills that were disqualified or repriced, outcomes descriptions, and audit trails, for example.

Although the exemplary network environment 100 with the bill review engine device 102, bill review management device 104, developer device 106, business analyst device 108, carrier device 112, and communication network(s) 110 and 114 are described and illustrated herein, other types and/or numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art(s).

One or more of the devices depicted in the network environment 100, such as the bill review engine device 102, bill review management device 104, developer device 106, business analyst device 108, or carrier device 112, for example, may be configured to operate as virtual instances on the same physical machine. In other words, one or more of the bill review engine device 102, bill review management device 104, developer device 106, business analyst device 108, or carrier device 112 may operate on the same physical device rather than as separate devices communicating through communication network(s) 110 and 114. Additionally, there may be more or fewer bill review engine devices, bill review management devices, developer devices, business analyst devices, or carrier devices than illustrated in FIG. 1.

In addition, two or more computing systems or devices can be substituted for any one of the systems or devices in any example. Accordingly, principles and advantages of distributed processing, such as redundancy and replication also can be implemented, as desired, to increase the robustness and performance of the devices and systems of the examples. The examples may also be implemented on computer system(s) that extend across any suitable network using any suitable interface mechanisms and traffic technologies, including by way of example only wireless networks, cellular networks, PDNs, the Internet, intranets, and combinations thereof.

The examples may also be embodied as one or more non-transitory computer readable media having instructions stored thereon for one or more aspects of the present technology, such as the memory 202 of the bill review engine device 102, as described and illustrated by way of the examples herein. The instructions in some examples include executable code that, when executed by one or more processors, such as the processor(s) 200 of the bill review engine device 102, cause the processors to carry out steps necessary to implement the methods of the examples of this technology that are described and illustrated herein.

An exemplary method for customized rule engines for automated bill review will now be described with reference to FIGS. 3-11. Referring more specifically to FIG. 3, a flowchart of an exemplary method for generating reusable logical patterns and deploying rules generated from the reusable logical patterns is illustrated. In step 300 in this example, the bill review engine device 102 determines whether a request is received to generate a new reusable logical pattern. In this example, the bill review engine device 102 facilitates generation of reusable logical patterns via an interface provided in response to a request from the developer device 106, although other methods of obtaining the reusable logical patterns can also be used. If the bill review engine device 102 determines that a request to generate a new reusable logical pattern has been received, then the Yes branch is taken to step 302.

In step 302, the bill review engine device 102 receives source code for a reusable logical pattern and stores the source code in the pattern library 208 associated with an identifier for the reusable logical pattern. The reusable logical pattern source code defines criteria, at least one action, and one or more template fields, which can be associated with the criteria and/or the action and can be populated by portion(s) of rule parameter data for rules built on top of the reusable logical pattern.

In one particular example in which the reusable logical pattern is used to generate rules associated with an adjustment module, the template field may be a percentage reduction of a baseline fee for an electronic medical bill, which can vary according to particular jurisdictions. For example, regulations of one state may require a 25% reduction in the baseline fee while regulations of another state may require a 35% reduction in the baseline fee. The reusable logical pattern source code in this example defines particular criteria including a medical procedure code and a modifier value.

The action in this example results in a calculation of an adjusted fee from the baseline fee according to the percentage reduction when the medical procedure code and modifier value criteria are met based on an analysis of the electronic medical bill content. Accordingly, the rules that can be created for the respective state jurisdictions share common logic along with distinct values for template field(s) and a common action (i.e., adjusting the baseline fee).

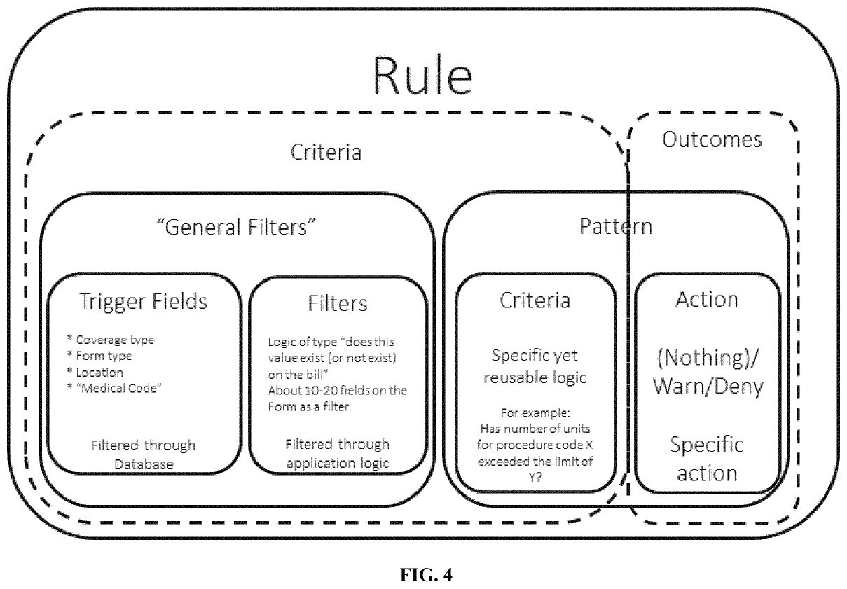

Referring more specifically to FIG. 4, a chart of exemplary rule contents is illustrated. A rule in this example includes criteria and outcomes with the outcomes corresponding to actions defined by a reusable logical pattern and rule criteria including general filters and pattern criteria of the reusable logical pattern. The general filters of the rule criteria can include trigger fields, such as coverage type, form type, location, or medical code, for example, that can correspond to data or contents associated with an electronic medical bill and can dictate whether a particular rule is triggered. The general filters can also include particular filters, such as whether value exists on an electronic medical bill that, if not present, may result in a disqualification of the electronic medical bill or a warning notification, for example.

The rule criteria further includes the pattern criteria that can be shared by any number of rules built on top of the reusable logical pattern and can include specific and reusable logic. In one example, the logic can evaluate whether a number of units for a particular medical procedure code has exceeded a specific limit. The number of units and limit may be template fields that are common among a number of rules (e.g., per state jurisdiction), but have values specific to each particular rule. The rule outcomes correspond to pattern actions, which can include generating a warning, denying or disqualifying an electronic medical bill, or setting or adjusting a baseline fee for an electronic medical bill, for example.

Referring back to FIG. 3, subsequent to receiving and storing the source code for a new reusable logical pattern, or if the bill review engine device 102 determines that a new pattern is not requested in step 300 and the No branch is taken, then the bill review engine device 102 proceeds to step 304. In step 304, the bill review engine device 102 determines whether a new rule is requested to be generated on top of a reusable logical pattern stored in the pattern library 208.

In this example, the bill review engine device 102 facilitates generation of a rule via an interface provided in response to a request from the business analyst device 108, although other methods of obtaining the rule can also be used. If the bill review engine device 102 determines that a request has not been received to generate a new rule, then the No branch is taken back to step 300, and the bill review engine device 102 effectively waits for a request to generate either a new pattern or a new rule. However, if the bill review engine device 102 determines that a request to generate a new rule has been received, then the Yes branch is taken to step 306.

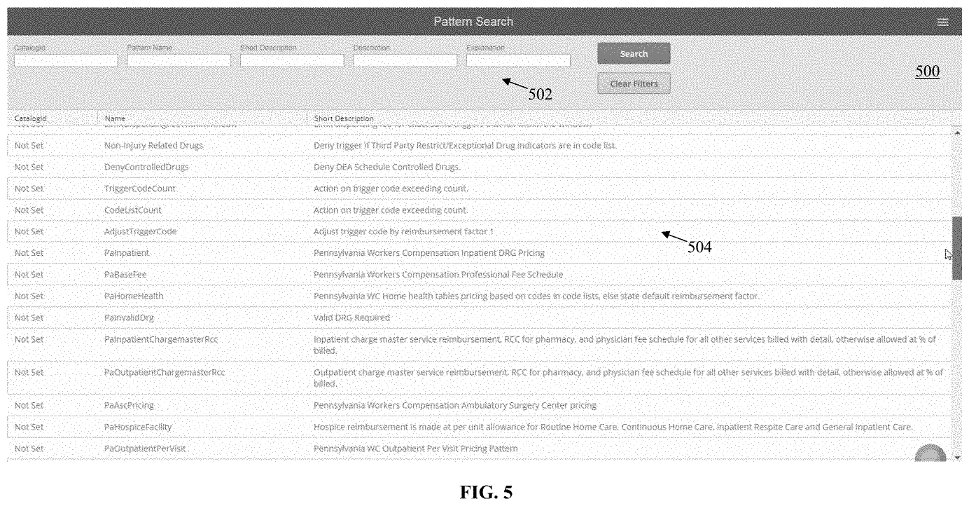

In step 306, the bill review engine device 102 receives a selection of a reusable logical pattern from the pattern library 208. Referring more specifically to FIG. 5, a screenshot of an exemplary pattern search interface 500 is illustrated. The pattern search interface in this example includes searchable fields 502 and a pattern listing 504 of identifiers (i.e., names) and a short description for reusable logical patterns maintained in the pattern store 208. Other methods for facilitating selection of a reusable logical pattern from which to define a rule can also be used in other examples. Additionally, in yet other examples, an existing rule from the rule store 214 can be accessed and cloned to initiate the generation of a new rule.

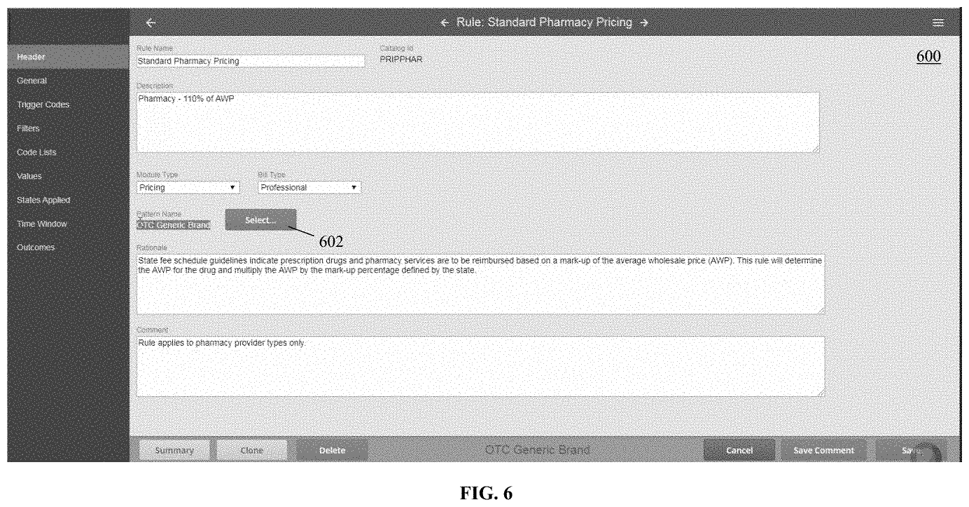

Referring back to FIG. 3, in step 308, the bill review engine device 102 obtains rule parameter data and generates, and stores in the rule store 214, a rule based on the reusable logical pattern selected in step 306 and the rule parameter data 308. In particular, the bill review engine device 102 can populate the template fields of the selected reusable logical pattern with portion(s) of the obtained rule parameter data. The rule parameter data can be obtained via an interface(s) provided to the business analyst device 108, for example, although the rule parameter data can also be obtained in other ways. Exemplary rule parameter interfaces will not be described with reference to FIGS. 6-10.

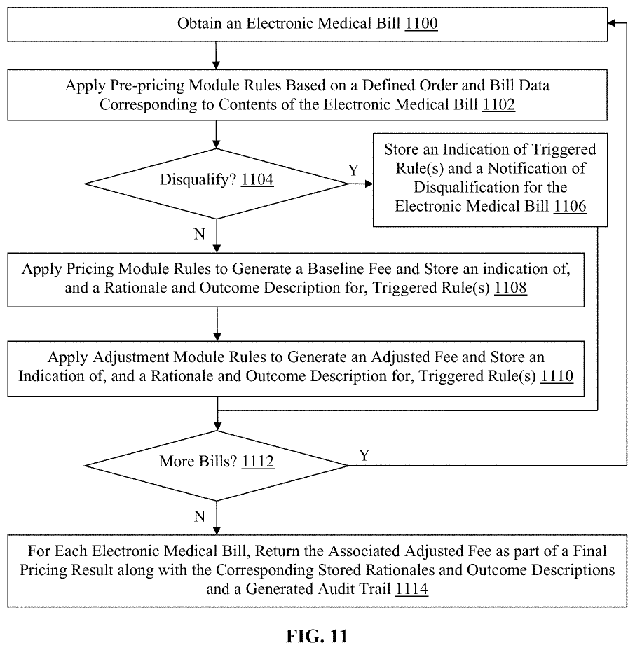

Referring more specifically to FIG. 6, a screenshot of an exemplary rule header interface 600 is illustrated. A user of the business analyst device 108 can input rule parameter data into fields of the rule header interface 600, such as a rule name, description, association module, bill type, rationale, and/or any additional comment. The associated module can relate to a collection of rules that, when triggered, are capable of performing a same category of actions. Exemplary pre-pricing, pricing, and adjustment modules are described and illustrated in more detail later with reference to FIG. 11.

The rationale in this example includes a narrative, human-readable description of the reasoning behind the rule, such as the particular state guideline, as well as the purpose of the rule, such as determining a standard pharmacy pricing for a drug based on an average wholesale price (AWP) and a mark-up percentage defined by a state regulation. Other rationales and other fields corresponding to other types of rule parameter data can also be included in the rule header interface 600 in other examples. Optionally, the rule header interface 600 includes a select button 602 that displays the pattern search interface 500, for example.

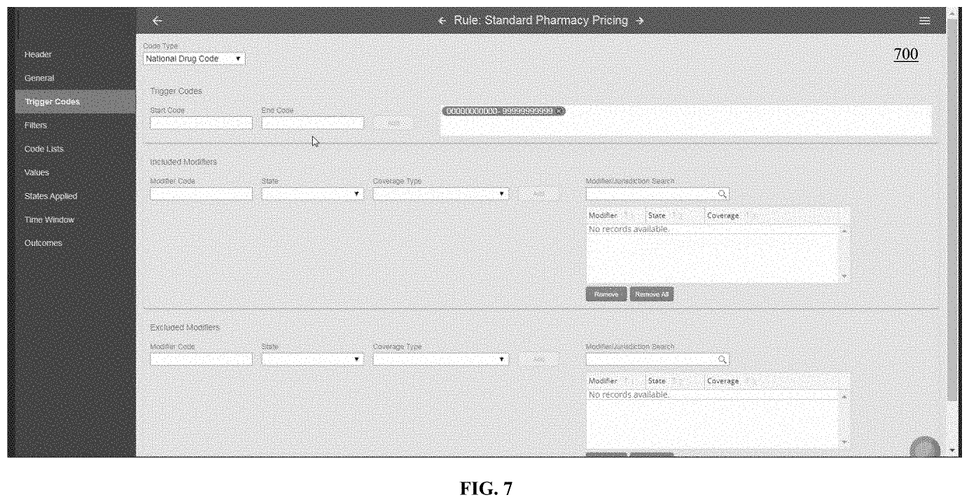

Referring more specifically to FIG. 7, a screenshot of an exemplary rule trigger code interface 700 is illustrated. A user of the business analyst device 108 can input rule parameter data into fields of the rule trigger code interface 700, such as a medical procedure or drug code or code range for which the new rule being defined will be triggered. The rule trigger code interface 700 in this particular example also includes fields to facilitate input of included and excluded modifiers, including the modifier code, state, and coverage type, although other types of modifier or rule trigger code fields can also be included in the rule trigger code interface 700 in other examples.

In previous systems the same triggering codes were repeated throughout the rules that all trigger on the same section of codes (for example, surgical codes 10000-69999 excluding several codes that should not be included in that range). When a new code is created, it was necessary to modify some or all of the related rules to reflect the new code. But in the disclosed embodiments, the codes are stored in a separate data dictionary that is accessed by the rule trigger code interface 700. The interface 700 then may allow the user to select a given attribute which they want to fire upon—such as “Surgery” or “Durable Medical Equipment”—and the interface 700 may respond by accessing the code as an attribute in a table in the data dictionary. A significant advantage of this approach is that the related rules, which may number in the thousands, can be updated without requiring new programing or new rule creation.

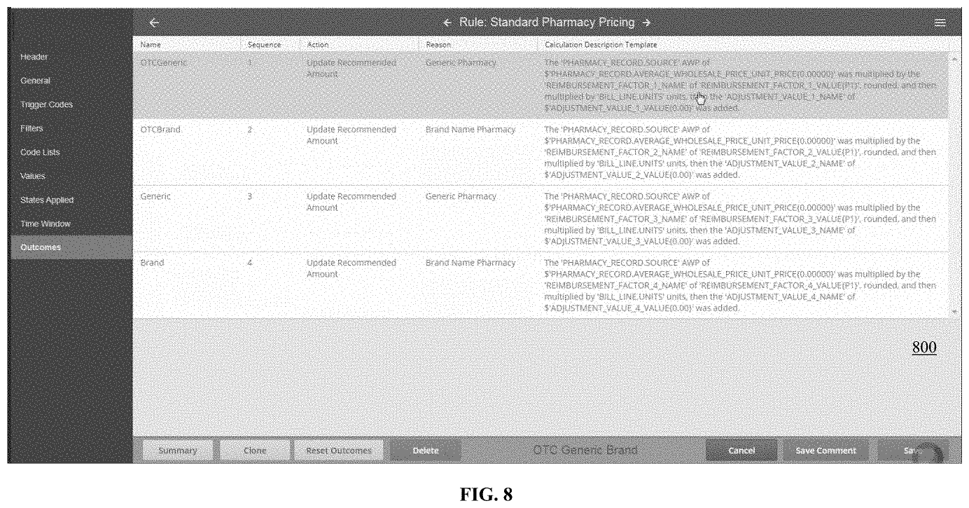

Referring more specifically to FIG. 8, a screenshot of an exemplary rule outcome interface 800 is illustrated. A user of the business analyst device 108 can input and/or view predefined rule parameter data into fields of the rule outcome interface 800, such as an outcome description for the rule being defined. In previous systems, a pattern was associated with only one outcome. But in the disclosed embodiments, a single pattern may be associated with multiple outcomes for added flexibility. In the example illustrated in FIG. 8, the rule relates to standard pharmacy pricing and there are four outcomes for generating an adjusted fee for a drug based on whether the drug is an over-the-counter (OTC) generic or a brand drug.

The rule outcome interface 800 includes a calculation description template with fields that are populated with database data when the rule is triggered to generate a human-readable narrative that can be returned as a result of the review of an associated electronic medical bill. In previous systems these calculation descriptions were hard-coded or not flexible. But in the disclosed embodiments, these calculation templates now allow the same pattern to read out differently depending on the rule parameters and individual system calculations and user modifications. Therefore, a user can create meaningful titles and user-friendly descriptions that say completely different things even though the same logical pattern is being used. Accordingly, the outcome description provides a recipient or source of an electronic medical bill (e.g., an insurance carrier or payer user of the carrier device 112) with a human-readable basis for the outcome, such as specifically how the baseline fee for the electronic medical bill. Other types of rule outcome fields can also be included in the rule outcome interface 800 in other examples.

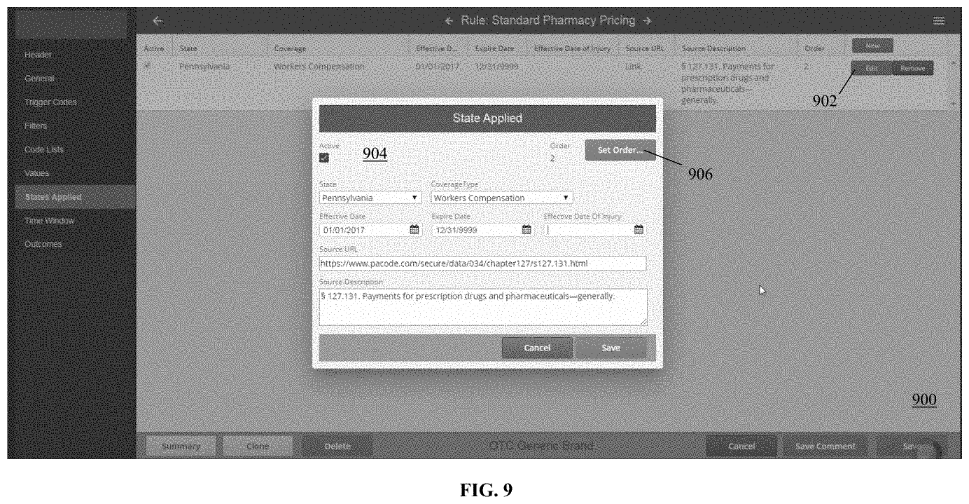

Referring more specifically to FIG. 9, a screenshot of an exemplary rule jurisdiction interface 900 is illustrated. A user of the business analyst device 108 can input rule parameter data into fields of the rule jurisdiction interface 900, such as the state, coverage type, effective date, source description, and source uniform resource locator (URL), for example, although another type or number of rule parameter data can also be obtained via the rule jurisdiction interface 900 in other examples.

In this particular example, selection of an edit button 902 on the rule jurisdiction interface 900 can generate an applied state overlay 904 that can facilitate input or edit of rule parameter data. The source description in this example includes a text narrative of the Pennsylvania regulation upon which the rule being generated is based and which is the source of the adjusted fee calculation that is the action resulting from the triggering of the rule. The applied state overlay 904 in this example also includes a set order button 906 that allows a user of the business analyst device 108, for example, to define an order of the application of the rule being generated among all the rules that may be tested for a particular state, for example, to determine whether they are triggered by the contents of electronic medical bill(s) being reviewed.

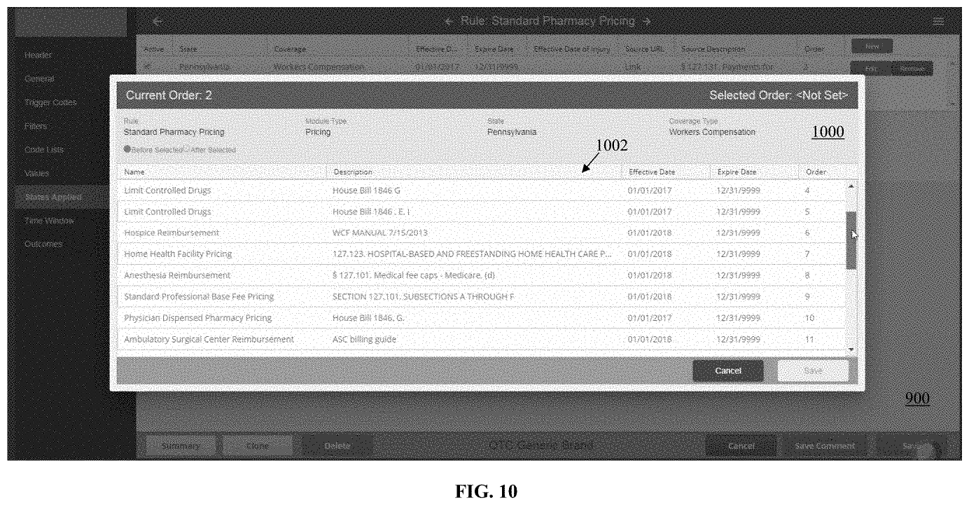

Referring more specifically to FIG. 10, a screenshot of an exemplary rule order overlay 1000 is illustrated. The rule order overlay 1000 can be generated upon selection of the set order button 906 of the applied state overlay 904, for example, although the rule order overlay 1000 can also be generated in other ways in other examples. The rule order overlay 1000 including a rule list 1002 that identifies a subset of the rules in the rule store 214, such as the pricing module rules for Pennsylvania associated with a workers compensation type of electronic medical bill, for example. With the rule list, a user of the business analyst device 108, for example, can advantageously adjust the hierarchy or order of the applied rules. This innovative feature allows a subject matter expert to change the rule sequence, for example when new government rules require changing the hierarchy. In some embodiments, the rule may inherit partial logic from existing patterns for new rule logics. While exemplary rule parameter data has been described and illustrated herein with reference to FIGS. 5-10, other rule parameter data including filters, values, and time windows, for example, can also be obtained via one or more provided interfaces in other examples.

Referring back to FIG. 3, in step 310, the bill review engine device 102 optionally executes the rule generated and stored in step 308 against test case scenarios for validation. The test case scenarios can include a set of electronic medical bills having known outcomes, such as disqualification or a pre-determined adjusted fee or final pricing amount, for example. The execution of the rule can be performed against the test case scenarios as described and illustrated in more detail later with reference to FIG. 11 with respect to a live deployment of the rule engine that incorporates the new rule. In other examples, the new rule can be tested or validated in other ways.

In step 312, the bill review engine device 102 deploys the validated rule, optionally as associated with a particular module or set of rules having a same associated category. The rule can be deployed by marking the rule as active or transiting the rule into a live rule store 214, for example, although deployment can also be facilitated in other ways. In iterations in which the rule is unable to be validated, one or more of steps 304-310 can be repeated in order to address the deficiency with respect to the new rule. Subsequent to deploying the rule, the bill review engine device 102 proceeds back to step 300 in this example. In other examples, one or more of steps 300-312 can be performed in parallel or in a different order.

Referring more specifically to FIG. 11, a flowchart of an exemplary method for executing an improved rule engine for automated electronic medical bill review is illustrated. In step 1100 in this example, the bill review engine device 102 obtains an electronic medical bill. The electronic medical bill can be obtained from the bill review management device 104 hosting a calling application that uses an application programming interface (API), for example, to communicate the electronic medical bills via the communication network(s) 110. Accordingly, any number of calling applications on any number of bill review management devices can utilize the bill review engine device 102 in this example. In other examples, the bill review engine device 102 can be integrated with the bill review management device 104, and other configurations can also be used.

Optionally, the electronic medical bill obtained in step 1100 can be part of a batch of electronic medical bills associated with an insurance carrier or payer that is associated with the carrier device 112. The electronic medical bills can be retrieved by the bill review management device 104 via an interface provided to the carrier device 112 and the communication network(s) 114, or the bill review management device 104 can obtain the electronic medical bills from a server or other device coupled to the bill review management device 104 via the communication network(s) 114, for example. The electronic medical bills can be in a portable data file (PDF) or other graphical format requiring pre-processing, such as optical character recognition (OCR) or another type of graphical analysis, for example, or the electronic medical bills can be processed prior to being obtained in step 1100, or obtained in a digital format, such that data reflecting content of the electronic medical bills is obtained directly in step 1100.

In step 1102, the bill review engine device 102 applies pre-pricing module rule(s) based on a defined order and bill data corresponding to the contents of the electronic medical bill obtained in step 1100. The pre-pricing module rules in this example are applied in an order that can be established as described and illustrated in more detail earlier with reference to FIGS. 9-10, for example. Additionally, the pre-pricing module rules include rules associated with a pre-pricing result or action (e.g., disqualification).

For example, if a pre-pricing module rule is triggered because the electronic medical bill indicates that it relates to a prepackaged drug but does not include an original national drug code (NDC), the action for the triggered rule may require disqualification of the electronic medical bill because it lacks an essential value for determining the appropriate associated baseline or adjusted fee according to a particular jurisdictional regulation. Other types of pre-pricing module rules can also be used in other examples.

In step 1104, the bill review engine device 102 determines whether the electronic medical bill should be disqualified based on the action associated with a triggered pre-pricing module rule. If the bill review engine device 102 determines that the electronic medical bill should be disqualified, then the Yes branch is taken to step 1106.

In step 1106, the bill review engine device 102 stores a notification of disqualification for the electronic medical bill along with an indication of the pre-pricing module rule(s) that were triggered. In some examples, the notifications and other information (e.g., final pricing amounts and outcome descriptions) to be returned can be queued and sent in a batch in response to a request to review a batch of electronic medical bills received from the bill review management device 104 in step 1100. Referring back to step 1104, if the bill review engine device 102 determines that the electronic medical bill should not be disqualified based on the application of the pre-pricing module rule(s), then the No branch is taken to step 1108.

In step 1108, the bill review engine device 102 applies pricing module rule(s) to the bill data for the electronic medical bill to generate a baseline fee for the electronic medical bill. Accordingly, the bill review engine device 102 applies rule(s) in the rule store 214 that are identified as associated with the pricing module to the bill data for the electronic medical bill to determine whether the rules are triggered. For example, a rule can be triggered based on a jurisdiction, provider type, and/or medical code (e.g., procedure or drug code) in the bill data, although a match of any other type or combination of rule parameter data can also result in triggering a pricing module rule in other examples. In this example, the pricing module rules associated with a particular portion of the rule parameter data, such as the jurisdiction, can be applied in the defined order, as described and illustrated in more detail earlier.

In step 1108, the bill review engine device 102 also stores in a queue or other data structure, an indication of the pricing module rule(s) that were triggered along with at least the rationale and outcome description for the pricing module rule(s) that were triggered in this example. The rationale and outcome description can be associated with the triggered pricing module rule(s) as part of parameter data obtained and stored as described and illustrated in more detail earlier with reference to FIGS. 6 and 8.

In step 1110, the bill review engine device 102 applies adjustment module rule(s) to generate an adjusted fee for the electronic medical bill. The bill review engine device 102 in this example applies rules in the rule store 214 that are identified as associated with the adjustment module to the bill data for the electronic medical bill to determine whether the rules are triggered. The bill review engine device 102 also stores an indication of the adjustment module rule(s) that were triggered and the rationale and outcome description associated with the adjustment module rule(s) that were triggered in this example.

An adjustment module rule can be triggered based on a jurisdiction, medical code, and/or particular modifier value in the bill data, for example, although a match of any other type or combination of rule parameter data can also result in triggering an adjustment module rule in other examples. The triggered adjustment module rule(s) in this example can have an associated action that multiplies the baseline fee by an adjustment value to generate the adjusted fee. In this example, the rules associated with a particular portion of the rule parameter data, such as the jurisdiction, can be applied in the defined order, as described and illustrated in more detail earlier.

While exemplary pre-pricing, pricing, and adjustment modules are used in the example described and illustrated with reference to FIG. 11, another number and/or type of module(s) can also be used in other examples. Subsequent to applying the adjustment module rule(s) in step 1110, or storing a notification of disqualification in step 1106, the bill review engine 102 proceeds to step 1112.

In step 1112, the bill review engine device 102 determines whether there are more electronic medical bills in the batch of electronic medical bills optionally received in step 1100. If the bill review engine 102 determines that there is at least one additional electronic medical bill that currently requires processing, then the Yes branch is taken back to step 1100, and steps 1100-1110 are repeated for the additional electronic medical bill(s). Alternatively, if the bill review engine 102 determines in step 1112 that there are no additional electronic medical bills that currently require processing, then the No branch is taken to step 1114.

In step 1114, the bill review engine device 102 returns a final pricing result, which may include the queued disqualification notifications (e.g., denied, warned, returned) and/or adjusted fee determined in step 1110 for each of the electronic medical bills in the batch received in step 1100. The bill review engine device 102 also returns the stored rationale and outcome description for each of the electronic medical bills not disqualified. The returned data can be sent to the calling application hosted by the bill review management device 104, for example, and provided by the bill review management device 104 to the carrier device 112 via an interface and the communication network(s) 114. These data may include industry or regulatory citation, source details with hyperlinks to support the determination, full end-user explanation of each rule fired, calculation details using meaningful end-user terms per each rule fired, and the like,

Accordingly, a user of the carrier device 112 can efficiently determine and process relatively large volumes of electronic medical bills using an interface provided by the bill review management device 104 and the information returned by the bill review engine device 102. The bill review engine device 102 advantageously returns outcome descriptions and rationales in a human-readable format that allows a review to determine how an electronic medical bill was repriced and confirm the accuracy of the output from the bill review engine device 102. Additionally, with the indication of each of the rule(s) that were triggered for the various modules, the bill review engine 102 can advantageously provide an audit trail to a graphical user interface made accessible by the bill review management device 104 to the carrier device 112. The audit trail may be created as each bill is processed, and may be exported to an audit log as one of the bill review results. Each audit trail ratio which rules were fired, which calculations were performed, and the outcome of each. With the audit trail, the user of the carrier device 112 can compare older versions of the same rule to track a detailed history across the entire system.

As illustrated and described by way of the examples herein, this technology utilizes reusable logical patterns to generate rules that facilitate a customized rule engine for automated electronic medical bill review. With this technology, rules based upon stored reusable logical patterns can be generated relatively quickly using graphical user interface(s), without requiring additional source code development, and by business analysts that understand the impact of a change in a government regulation, but do not have software development expertise. Accordingly, this technology solves the technological problem of rapid rule development and deployment for rule engines that process electronic medical bills in order to improve the rule engine accuracy. The improved rule engine of this technology also advantageously maintains an audit trail and human-readable outcome descriptions for triggered rules to provide improved reporting of the results of automated electronic medical bill reviews.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A method for automated medical bill review, the method comprising:
receiving, by a bill review engine device, a selection of a reusable logical pattern from a pattern library, wherein the reusable logical pattern comprises source code defining at least one action and one or more template fields;
populating, by the bill review engine device, the template fields based on received rule parameter data to generate and store a rule, wherein the rule parameter data comprises one or more triggering codes;
applying, by the bill review engine device, the rule to contents of a received electronic medical bill to determine when the rule is triggered based on the triggering codes; and
returning, by the bill review engine device, a pricing result for the electronic medical bill to a source of the electronic medical bill, when the determination indicates that the rule is triggered, wherein the pricing result is determined based on the action.

2. The method of claim 1, wherein the rule is one of a plurality of rules applied to the electronic medical bill and the method further comprises providing, by the bill review engine device, a generated audit trail via a graphical user interface accessible by the source of the electronic medical bill, wherein the audit trail comprises at least an indication of each of a subset of the rules that were triggered during the application of the rules to the electronic medical bill.

3. The method of claim 1, wherein the rule parameter data comprises a jurisdiction, the action comprises generating a baseline or adjusted fee for the electronic medical bill that complies with one or more regulations for the jurisdiction, and the pricing result comprises the baseline or adjusted fee.

4. The method of claim 3, further comprising reordering, by the bill review engine device, the rule within a hierarchy comprising a plurality of rules applicable to the jurisdiction, wherein the rules are applied in an order according to the hierarchy.

5. The method of claim 1, wherein the source code for the reusable logical pattern further defines at least one outcome description template and the method further comprises returning, by the bill review engine device, an outcome description narrative, generated by populating the outcome description template, to the source of the electronic medical bill along with the pricing result, when the determination indicates that the rule is triggered.

6. The method of claim 1, further comprising deploying, by the bill review engine device, the rule in a live environment subsequent to generating the rule and prior to applying the rule, when the rule is validated based on an execution of the rule against a plurality of test electronic medical bills with known pricing amounts.

7. A bill review engine device, comprising memory comprising programmed instructions stored thereon and one or more processors configured to execute the stored programmed instructions to:
receive a selection of a reusable logical pattern from a pattern library, wherein the reusable logical pattern comprises source code defining at least one action and one or more template fields;
populate the template fields based on received rule parameter data to generate and store a rule, wherein the rule parameter data comprises one or more triggering codes;
apply the rule to contents of a received electronic medical bill to determine when the rule is triggered based on the triggering codes; and
return a pricing result for the electronic medical bill to a source of the electronic medical bill, when the determination indicates that the rule is triggered, wherein the pricing result is determined based on the action.

8. The bill review engine device of claim 7, wherein the rule is one of a plurality of rules applied to the electronic medical bill and the processors are further configured to execute the stored programmed instructions to provide a generated audit trail via a graphical user interface accessible by the source of the electronic medical bill, wherein the audit trail comprises at least an indication of each of a subset of the rules that were triggered during the application of the rules to the electronic medical bill.

9. The bill review engine device of claim 7, wherein the rule parameter data comprises a jurisdiction, the action comprises generating a baseline or adjusted fee for the electronic medical bill that complies with one or more regulations for the jurisdiction, and the pricing result comprises the baseline or adjusted fee.

10. The bill review engine device of claim 9, wherein the processors are further configured to execute the stored programmed instructions to reorder the rule within a hierarchy comprising a plurality of rules applicable to the jurisdiction, wherein the rules are applied in an order according to the hierarchy.

11. The bill review engine device of claim 7, wherein the source code for the reusable logical pattern further defines at least one outcome description template and the processors are further configured to execute the stored programmed instructions to return an outcome description narrative, generated by populating the outcome description template, to the source of the electronic medical bill along with the pricing result, when the determination indicates that the rule is triggered.

12. The bill review engine device of claim 7, wherein the processors are further configured to execute the stored programmed instructions to deploy the rule in a live environment subsequent to generating the rule and prior to applying the rule, when the rule is validated based on an execution of the rule against a plurality of test electronic medical bills with known pricing amounts.

13. A non-transitory machine readable medium having stored thereon instructions for automated medical bill review comprising executable code that, when executed by one or more processors, causes processors to:
receive a selection of a reusable logical pattern from a pattern library, wherein the reusable logical pattern comprises source code defining at least one action and one or more template fields;
populate the template fields based on received rule parameter data to generate and store a rule, wherein the rule parameter data comprises one or more triggering codes;
apply the rule to contents of a received electronic medical bill to determine when the rule is triggered based on the triggering codes; and
return a pricing result for the electronic medical bill to a source of the electronic medical bill, when the determination indicates that the rule is triggered, wherein the pricing result is determined based on the action.

14. The non-transitory machine readable medium of claim 13, wherein the rule is one of a plurality of rules applied to the electronic medical bill and the executable code, when executed by the processors, further causes the processors to provide a generated audit trail via a graphical user interface accessible by the source of the electronic medical bill, wherein the audit trail comprises at least an indication of each of a subset of the rules that were triggered during the application of the rules to the electronic medical bill.

15. The non-transitory machine readable medium of claim 13, wherein the rule parameter data comprises a jurisdiction, the action comprises generating a baseline or adjusted fee for the electronic medical bill that complies with one or more regulations for the jurisdiction, and the pricing result comprises the baseline or adjusted fee.

16. The non-transitory machine readable medium of claim 15, wherein the executable code, when executed by the processors, further causes the processors to reorder the rule within a hierarchy comprising a plurality of rules applicable to the jurisdiction, wherein the rules are applied in an order according to the hierarchy.

17. The non-transitory machine readable medium of claim 13, wherein the source code for the reusable logical pattern further defines at least one outcome description template and the executable code, when executed by the processors, further causes the processors to return an outcome description narrative, generated by populating the outcome description template, to the source of the electronic medical bill along with the pricing result, when the determination indicates that the rule is triggered.

18. The non-transitory machine readable medium of claim 13, wherein the executable code, when executed by the processors, further causes the processors to deploy the rule in a live environment subsequent to generating the rule and prior to applying the rule, when the rule is validated based on an execution of the rule against a plurality of test electronic medical bills with known pricing amounts.
